(19) United States
(12) Patent Application Publication
(10) Pub. No.: US 2020/0155867 A1
(43) Pub. Date: May 21, 2020

(54) Device for its use to destroy cancerous cells

(71) Applicant: Mohamed A. Basiony, Kenmore, WA (US)

(72) Inventor: Mohamed A. Basiony, Kenmore, WA (US)

(21) Appl. No.: 16/675,996

(22) Filed: Nov. 6, 2019

Related U.S. Application Data
(60) Provisional application No. 62/768,905, filed on Nov. 17, 2018.

(51) Int. Cl. A61N 5/06; A61M 5/31; A61M 5/315; A61M 5/32

(52) U.S. Cl. CPC A61N 5/062; A61M 5/3137; A61M 5/31513; A61M 5/3293; A61M 2005/3139; A61N 2005/0661; A61N 2005/0654; A61N 2005/0652; A61M 2205/32; A61M 5/329

(57) ABSTRACT

A device to be used to destroy cancerous cells comprises a modified syringe with an ultraviolet radiation source. A modified syringe comprises a hollow syringe barrel including a fluid chamber, a plunger movable on advanced and retracted position slidably disposed within a fluid chamber, an ultraviolet radiation source that is fixed longitudinally through hollow syringe barrel and a plunger.

An ultraviolet radiation source fixation seal including two apertures disposed within a fluid chamber to permit a photoactive agent to flow from its container into a fluid chamber through the two apertures. Then, a photoactive agent in a fluid chamber is activated with an ultraviolet radiation that is emitted from an ultraviolet radiation source before it is injected directly to a cancerous cells' location using a modified syringe which is acting as an ordinary syringe.

### BACKGROUND

Therapies such as a photodynamic therapy (PDT), is a treatment that uses special drugs, called photoactive (photosensitizing) agents, along with light to kill cancerous cells. Photoactive agents may only work after they have been activated or “turned on” by certain kinds of light.

Depending on a part of a body being treated, a photoactive agent (drug) is either put into a bloodstream through a vein or put on the skin. Over a certain amount of time a photoactive agent (drug) is absorbed by cancerous cells. Then light is applied to the area to be treated. A period of time between when a radioactive agent (drug) is given and when the light is applied is called the drug-to-light interval. It can be anywhere from a couple of hours to a couple of days, depending on a photoactive agent (drug) used.

Without activation, a photoactive agent may have a little or no effect on cells' chemistry, however upon activation, a photoactive agent is induced to form a permanent photo-adduct on tumor cells and chemically destruction of adducted constituent may be assumed.

In U.S. Pat. No. 4,649,151 to Dougherty, et. al. discloses the utilization of tumor selective photoactive agents (i.e. mixtures of porphyrins) in the localization and treatment of neoplastic tissue such as tumors or cancers in patients for its affinity toward tumor cells. In U.S. Pat. No. 4,428,744 to Edelson, et. al. discloses the utilization of psoralens as photoactive chemical agents that have affinity for DNA.

Scientists from Newcastle University, UK may use photoactivated antibodies as photoactive agents and they may aim to be used with tumors close to the skin, such as breast cancer, and for any cancers accessible to a light probe. These may include those of the digestive system, such as stomach and bowel cancer, and those of the genito-urinary system, such as ovarian cancer (Daily Mail-heath, Oct. 29, 2007).

It must be pointed out that in prior art, the photoactive agents may be injected into the blood stream or on in the skin and then activated by the light.

There is a need for a simple and less cost device that may facilitate the photodynamic therapy, to may save a patient time, and to may target directly the cancerous cells. Furthermore, it may improve the prior art limitations related to tumor location as it may be used to treat large cancers that have grown deeply into the skin or other organs.

### SUMMARY

Accordingly, a device comprises a modified syringe with an ultraviolet radiation source is described to may address the above issues.

A modified syringe comprises a hollow syringe barrel including a fluid chamber, a plunger movable on advanced and retracted position slidably disposed within a fluid chamber, an ultraviolet radiation source fixed longitudinally through hollow syringe barrel and a plunger.

An ultraviolet radiation source fixation seal including two apertures disposed within a fluid chamber to permit a photoactive agent to flow from its container into a fluid chamber through the two apertures. Then, a photoactive agent in a fluid chamber is activated with an ultraviolet radiation that is emitted from an ultraviolet radiation source before it is injected directly to a cancerous cells location using a modified syringe which is acting as an ordinary syringe.

### BRIEF DESCRIPTION OF THE DRAWINGS

The accompanying drawings, which are incorporated in, and constitute a part of the specification, illustrate or exemplify embodiment of the present implementation and, together with the description, generally explain the principles and features of the present implementation. The drawings are briefly described as follows:

FIG. 1 is a cross-sectional side view of the device that comprises a modified syringe and an ultraviolet radiation source according to the present disclosure.

FIG. 1a is a cross-sectional view along line 1a-1a of the modified syringe of FIG. 1 according to the present disclosure.

FIG. 2 is a schematic view of the device of FIG. 1 in a step one of its operation according to the present disclosure.

FIG. 3 is a schematic view of the device of FIG. 1 in a step two of its operation according to the present disclosure.

FIG. 4 is a schematic view of the device of FIG. 1 in a step three of its operation according to the present disclosure.

### DETAILED DESCRIPTION

The following detailed description illustrates the principal of the disclosure by way of example not by way of limitation. While a reference use of the present disclosure describes a device to be used to destroy cancerous cells, as those of ordinary skill in the art will readily understand. Also, it will be understood that the device may also be used for other types of treatments such as to enhance the immune system, consequently, the scope of the implementation is not to be limited by the field to which the implementation is applied.

The device according to present disclosure comprises a modified syringe with an ultraviolet radiation source.

An ultraviolet radiation source may use an ultraviolet spectrum that is suitable with a photoactive agent that is being used. In U.S. Pat. No. 4,878,891 to Judy et. al. discloses a range of an ultraviolet wavelengths from about 350 nm to about 700 nm. In U.S. Pat. No. 4,428,744 to Edelson et. al. discloses a wavelength range of about 200 nm to about 400 nm. An ultraviolet radiation source may have a lamp or light emitting diodes to may provide a specific wavelength of radiation known to be clinically effective to activate the radioactive agent.

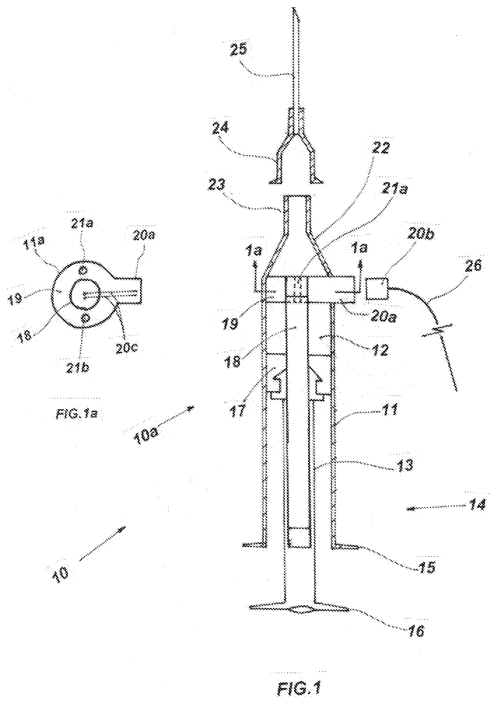

FIG. 1 illustrates a cross-sectional side view of a device 10 that comprises a modified syringe 10a that may be capable of functioning as an air tight syringe with an ultraviolet radiation source 18 wherein, a syringe cylindrical body 11, a hollow cylindrical syringe barrel 14 having a plunger element 13 slidably disposed therein. While the outer surface of a syringe cylindrical body 11 may be coated with a protective material to protect the user from being exposed to an ultraviolet radiation, the inner surface may be coated with a refractive material to reflect the radiation into a photoactive agent.

In particular, a modified syringe 10a may comprise a hollow cylindrical syringe barrel 14 including a fluid channel 12 and a plunger element 13 that may be movable between an advanced and retracted position slidably disposed within a fluid chamber 12. An ultraviolet radiation source 18 may be fixed longitudinally through a hollow cylindrical syringe barrel 14 and a plunger element 13.

An ultraviolet radiation source seal 19 may be used to fix an ultraviolet radiation source 18 at the center of a hollow cylindrical syringe barrel 14. An ultraviolet radiation source seal 19 may include two apertures 21a and 21b that may be disposed within a fluid chamber 12 to permit a photoactive agent to flow from its container into a fluid chamber 12 through apertures 21a and 21b to be activated by an ultraviolet radiation from an ultraviolet radiation source 18 for its activation process.

A modified syringe 10a may have a conically shaped end 22 and a syringe tip 23 that support a needle hub 24 of a needle 25. A finger grasping element 15 may be formed on opposite end of a syringe tip 23. A plunger element 13 may have a plunger seal 17 while the opposite end includes a thumb element 16 as in an ordinary syringe.

An ultraviolet radiation source seal 19 may extended to form a socket 20a that is connected to a socket 20b to power an ultraviolet radiation source 18 via a cable 26 and leads 20c.

FIG. 1a is across-sectional view along line 1a-1a, wherein a syringe outer surface 11a, an ultraviolet radiation source seal 19, an ultraviolet radiation source 18, two apertures 21a and 21b, a power socket 20a and power leads 20c.

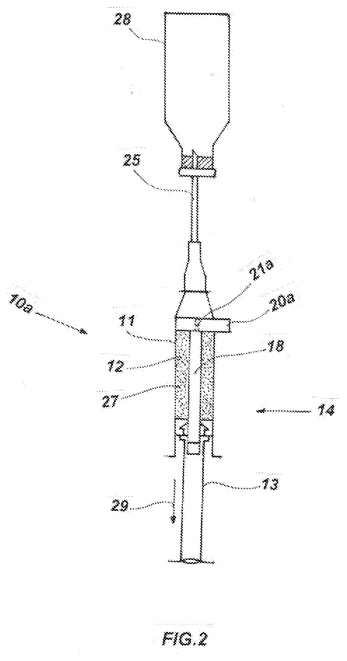

In use the device may have three steps. In first step as in FIG. 2, a modified syringe 10a that may have a syringe cylindrical body 11 is aspirated by the retraction of a cylindrical plunger element 13 as per arrow 29 within the hollow cylindrical syringe barrel 14, permitting a photoactive agent 27 to pass from a photoactive agent container 28 into a fluid chamber 12 through a needle 25 and apertures 21a and 21b (not shown for simplicity). The retraction of a cylindrical plunger element 13 is continued until a fluid chamber 12 has a suitable amount of a photoactive agent 27 as in an ordinary syringe. In this step an ultraviolet radiation source 18 may not be activated as no power connected to a socket 20a. The rest of components are the same as in FIG. 1.

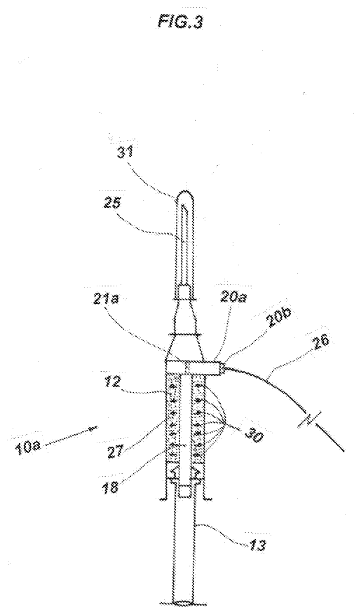

In step 2 of operation as in FIG. 3 an ultraviolet radiation source 18 within a modified syringe 10a is energized via a cable 26 and two sockets 20a and 20b and starts to emit an ultraviolet radiation 30 to activate a photoactive agent 27 inside a fluid chamber 12 for a certain period of time that is dependent on the type of an photoactive agent that is being used. So, with this implementation, the activation of a photoactive agent may be done outside the body to save the patient time and to may avoid injecting a photoactive agent in a blood stream and to may minimize a drug-to-light interval. To keep the device sterile a needle 25 may be covered by a sterile cover 31. The rest of components are the same as in FIG. 1

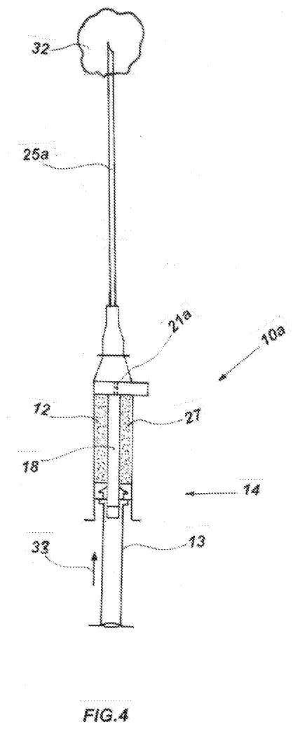

In step 3 of operation as in FIG. 4, a power is disconnected from an ultraviolet radiation source 18 within a modified syringe 10a and a plunger element 13 is advanced as per arrow 33 within a hollow cylindrical syringe barrel 14 to inject a photoactive agent 27 (that is inside a fluid chamber 12) through two apertures 21a and 21b (not shown for simplicity) and a special needle 25a directly to a cancerous cells' location 32. A special needle 25a may have different lengths and shapes based on cancerous cells locations. With this implementation, cancerous cells may be targeting directly by using special needles with different lengths and shapes, and we may be able to target large cancers that have grown deeply into the skin or other organs. The rest of components are the same as in FIG. 1.

1. A device comprising a modified syringe with an ultraviolet radiation source:
A modified syringe comprises a syringe cylindrical body and a hollow syringe barrel that is including a fluid chamber, a plunger element that is movable on advanced and retracted position slidably disposed within a fluid chamber. Said plunger has a plunger seal and its opposite end includes a thumb element as in an ordinary syringe. Said modified syringe has a syringe tip, a conically shaped end that support a needle hub with a needle. A finger grasping element is formed on opposite end of a syringe as in ordinary syringe; and
An ultraviolet radiation source is fixed longitudinally through said hollow syringe barrel and said syringe plunger via an ultraviolet radiation source fixation seal that is including two apertures disposed within a syringe fluid chamber. Said an ultraviolet radiation source seal is extended to form a power socket that is used to connect said ultraviolet radiation source to a power source.

2. The device of claim 1, wherein said modified syringe is capable of functioning as an air tight syringe as in an ordinary syringe.

3. The device of claim 1, wherein said ultraviolet radiation source includes a lamp or light emitting diodes.

4. A process, comprising three steps of operation:
In a step one of operation, said modified syringe is aspirated by the retraction of said cylindrical plunger element within said hollow cylindrical syringe barrel, permitting a photoactive agent to pass from a photoactive agent container into said fluid chamber through said needle and said apertures. The retraction of said cylindrical plunger element is continued until said fluid chamber has a suitable amount of said photoactive agent. In this step said ultraviolet radiation source is not activated as no power connected.
In a step two of operation, said ultraviolet radiation source within said modified syringe is energized and starts to emit an ultraviolet radiation to activate said photoactive agent inside said fluid chamber 12 for a certain period of time.
In a step three of operation, the power is disconnected from said ultraviolet radiation source and said plunger element is advanced within said hollow cylindrical syringe barrel to inject said photoactive agent through said two apertures and a special needle directly to a cancerous cells' location.

5. The process of claim 4, wherein said special needle has a different length and shapes.
